(19) United States
(12) Patent Application Publication
(10) Pub. No.: US 2026/0216534 A1
(43) Pub. Date: Jul. 30, 2026

(54) LINEAR ACCELERATOR PULSE RECORDING

(71) Applicant: ELEKTA BEIJING MEDICAL SYSTEMS CO., LTD., Beijing (CN)

(72) Inventor: Fan YI, Beijing (CN)

(21) Appl. No.: 19/143,309

(22) PCT Filed: Dec. 29, 2022
(86) PCT No.: PCT/CN2022/143303

§ 371 Date: Jun. 25, 2025

(51) Int. Cl. A61N 5/10; A61B 17/00; G16H 20/40; G16H 40/40

(52) U.S. Cl. CPC A61N 5/1075; G16H 20/40; G16H 40/40; A61B 2017/00159

(57) ABSTRACT

Methods, apparatus and systems are disclosed for analysing linac pulse signal samples to determine whether their shape is a standard one or not. When the shape is not a standard one, saving the raw data of the linac pulse signal samples, and, when the shape is a standard one, saving aggregated data that describes the linac pulse signal using less memory than the raw data.

This disclosure relates to apparatus, devices, systems, and approaches for radiotherapy, and in particular but without limitation to methods, apparatus and/or systems for linear accelerator, linac, pulse recording.

### BACKGROUND

Radiotherapy can be described as the use of ionising radiation, such as X-rays, to treat a human or animal body. Radiotherapy is commonly used to treat tumours within the body of a human or animal patient, or subject. In such treatments, ionising radiation is used to irradiate, and thus destroy or damage, cells which form part of the tumour.

The linear accelerators used for radiotherapy are highly complex machines having a significant number of complex interacting subsystems. User-friendly interfaces allow clinicians to easily control and monitor linac behaviour and may provide access to error or warning logs. However such error or warning logs are an after-effect or by product of the root cause of an issue, making it difficult to determine the actual conditions directly related to a performance degradation or failure.

### SUMMARY

An invention is set out in the claims.

### FIGURES

Specific examples are now described, by way of example only, with reference to the drawings, in which:

FIG. 1 shows a radiotherapy system;

FIG. 2 shows a pulse signal hub 205 that is arranged to record the triggered signals;

FIG. 3 shows a flowchart of a method of processing pulse signal information of a radiotherapy linear accelerator;

FIG. 4 shows an example output of a method of processing pulse signal information of a radiotherapy linear accelerator;

FIG. 5 shows a flowchart of a method of logging pulse signal abnormal waveforms;

FIG. 6 is a continuation of FIG. 5;

FIG. 7 shows a flowchart of a method of interacting with a central model for error logging; and

FIG. 8 is a continuation of FIG. 7.

### DETAILED DESCRIPTION

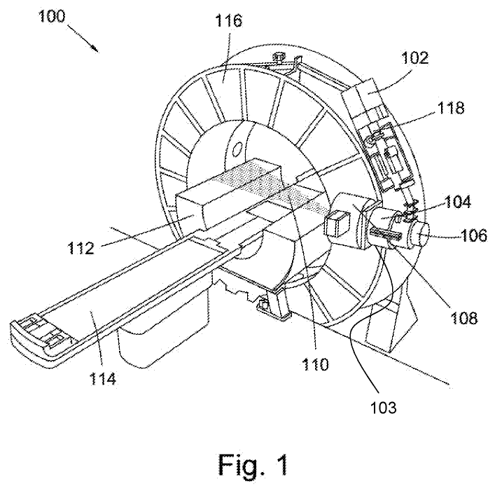

FIG. 1 shows a radiotherapy system, or device, suitable for delivering, and configured to deliver, a beam of radiation to a patient during radiotherapy treatment. The device and its constituent components will be described generally for the purpose of providing useful accompanying information for the present disclosure. The device shown in FIG. 1 is in accordance with the present disclosure and is suitable for use with the disclosed systems and apparatuses. While the device in FIG. 1 is an MR-linac (magnetic resonance linear accelerator), the implementations of the present disclosure may be any radiotherapy device, for example a linac (linear accelerator) device.

The device 100 shown in FIG. 1 is an MR-linac. The device 100 comprises both MR imaging apparatus 112 and radiotherapy (RT) apparatus which may comprise a linac device. The MR imaging apparatus 112 is shown in the diagram in a partially cut away perspective manner. In operation, the MR scanner produces MR images of the patient, and the linac device produces and shapes a beam of radiation and directs it towards a target region within a patient's body in accordance with a radiotherapy treatment plan. FIG. 1 does not show the usual ‘housing’ which would cover the MR imaging apparatus 112 and RT apparatus in a commercial setting such as a hospital.

The MR-linac device shown in FIG. 1 comprises a source 102 of radiofrequency (RF) waves, a waveguide 104, an electron source 106, a radiation source 103, a collimator 108 such as a multi-leaf collimator configured to collimate and shape the beam, MR imaging apparatus 112 (shown partially cut away), and a patient support surface 114. In use, the device would also comprise a housing (not shown) which, together with the ring-shaped gantry, defines a bore. The patient support surface 114 is moveable and can be used to support a patient and move them, or another subject, into the bore when an MR scan and/or when radiotherapy is to commence. The MR imaging apparatus 112, RT apparatus, and a patient support surface actuator are communicatively coupled to a controller or processor. The controller is also communicatively coupled to a memory device comprising computer-executable instructions which may be executed by the controller.

The RT apparatus comprises a radiation source 103 and a radiation detector (not shown). Typically, the radiation detector is positioned diametrically opposed to the radiation source 103. The radiation detector is suitable for, and configured to, produce radiation intensity data. In particular, the radiation detector is positioned and configured to detect the intensity of radiation which has passed through the subject. The radiation detector may also be described as radiation detecting means, and may form part of a portal imaging system.

The radiation source 103 may comprise a beam generation system. For a linac, the beam generation system may comprise a source 102 of RF waves, an electron source 106 such as an electron gun (or e-gun), and a waveguide 104. The radiation source 103 is attached to the rotatable gantry 116 so as to rotate with the gantry 116. In this way, the radiation source 103 is rotatable around the patient so that a treatment beam 110 can be applied from different angles around the gantry 116. In a preferred implementation, the gantry is continuously rotatable. In other words, the gantry can be rotated by 360 degrees around the patient, and in fact may continue to be rotated past 360 degrees. The gantry may be ring-shaped. In other words, the gantry may be a ring-gantry.

The source 102 of radiofrequency waves, such as a magnetron, is configured to produce radiofrequency waves. The source 102 of radiofrequency waves is coupled to the waveguide 104 via a circulator 118 and is configured to pulse radiofrequency waves into the waveguide 104. Radiofrequency waves may pass from the source 102 of radiofrequency waves through an RF input window and into an RF input connecting pipe or tube. The electron source 106 is also coupled to the waveguide 104 and is configured to inject electrons into the waveguide 104. In the electron source 106, electrons are thermionically emitted from a cathode filament as the filament is heated. The temperature of the filament controls the number of electrons injected. The injection of electrons into the waveguide 104 is synchronised with the pumping of the radiofrequency waves into the waveguide 104. The design and operation of the source 102 of radiofrequency waves, the electron source 106 and the waveguide 104 is such that the radiofrequency waves accelerate the electrons to very high energies as the electrons propagate through the waveguide 104.

The design of the waveguide 104 depends on whether the linac accelerates the electrons using a standing wave or travelling wave, though the waveguide typically comprises a series of cells or cavities, each cavity connected by a hole or ‘iris’ through which the electron beam may pass. The cavities are coupled in order that a suitable electric field pattern is produced which accelerates electrons propagating through the waveguide 104. As the electrons are accelerated in the waveguide 104, the electron beam path is controlled by a suitable arrangement of steering magnets, or steering coils, which surround the waveguide 104. The arrangement of steering magnets may comprise, for example, two sets of quadrupole magnets.

Once the electrons have been accelerated, they may pass into a flight tube. The flight tube may be connected to the waveguide by a connecting tube. This connecting tube or connecting structure may be called a drift tube. The electrons travel toward a heavy metal target which may comprise, for example, tungsten. Whilst the electrons travel through the flight tube, an arrangement of focusing magnets act to direct and focus the beam on the target.

To ensure that propagation of the electrons is not impeded as the electron beam travels toward the target, the waveguide 104 is evacuated using a vacuum system comprising a vacuum pump or an arrangement of vacuum pumps. The pump system is capable of producing ultra-high vacuum (UHV) conditions in the waveguide 104 and in the flight tube. The vacuum system also ensures UHV conditions in the electron gun. Electrons can be accelerated to speeds approaching the speed of light in the evacuated waveguide 104.

The radiation source 103 is configured to direct the treatment beam 110 of therapeutic radiation toward a patient positioned on the patient support surface 114. The radiation source 103 may therefore also be referred to as a therapeutic radiation source. The radiation source 103 may comprise a heavy metal target towards which the high energy electrons exiting the waveguide are directed. When the electrons strike the target, X-rays are produced in a variety of directions. A primary collimator may block X-rays travelling in certain directions and pass only forward travelling X-rays to produce the treatment beam 110. The X-rays may be filtered and may pass through a primary ion chamber and a secondary ion for dose measuring. The beam can be shaped in various ways by beam-shaping apparatus, for example by using the multi-leaf collimator 108, before it passes into the patient as part of radiotherapy treatment.

In some implementations, the radiation source 103 is configured to emit either an X-ray beam or an electron particle beam. Such implementations allow the device to provide electron beam therapy, i.e. a type of external beam therapy where electrons, rather than X-rays, are directed toward the target region as the therapeutic radiation. It is possible to ‘swap’ between a first mode in which X-rays are emitted and a second mode in which electrons are emitted by adjusting the components of the linac. In essence, it is possible to swap between the first and second mode by moving the heavy metal target in or out of the electron beam path and replacing it with a so-called ‘electron window’. The electron window is substantially transparent to electrons and allows electrons to exit the flight tube.

The subject or patient support surface 114 is configured to move between a first position substantially outside the bore, and a second position substantially inside the bore. In the first position, a patient or subject can mount the patient support surface. The patient support surface 114, and patient, can then be moved inside the bore, to the second position, in order for the patient to be imaged by the MR imaging apparatus 112 and/or imaged or treated using the RT apparatus. The bore may hence lie about a portion of space that is suitable for receiving at least a portion of a patient—a patient receiving space. The movement of the patient support surface is effected and controlled by a patient support surface actuator, which may be described as an actuation mechanism. Together, these components may be described as a patient positioning system, which may comprise other components. The actuation mechanism is configured to move the patient support surface in a direction parallel to, and defined by, the central axis of the bore. The terms subject and patient are used interchangeably herein such that the patient support surface can also be described as a subject support surface. The patient support surface may also be referred to as a moveable or adjustable couch or table.

The radiotherapy apparatus/device shown in FIG. 1 also comprises an optional MR imaging apparatus 112. The MR imaging apparatus 112 is configured to obtain images of a subject positioned, i.e. located, on the patient support surface 114. The MR imaging apparatus 112 may also be referred to as the MR imager. The MR imaging apparatus 112 may be a conventional MR imaging apparatus operating in a known manner to obtain MR data, for example MR images. The skilled person will appreciate that such an MR imaging apparatus 112 may comprise a primary magnet, one or more gradient coils, one or more receive coils, and an RF pulse applicator. The operation of the MR imaging apparatus is controlled by the controller.

The controller is a computer, processor, or other processing apparatus. The controller may be formed by several discrete processors; for example, the controller may comprise an MR imaging apparatus processor, which controls the MR imaging apparatus 110; an RT apparatus processor, which controls the operation of the RT apparatus; and a subject support surface processor which controls the operation and actuation of the patient support surface. The controller is communicatively coupled to a memory, e.g. a computer readable medium.

The linac device also comprises several other components and systems as will be understood by the skilled person. For example, in order to ensure the linac does not leak radiation, appropriate shielding is also provided.

Each time a dose of radiotherapy is to be given to a patient positioned on the patient support surface a trigger signal or signals cause a dose or radiation to be emitted from the radiation source. The trigger signal or signals is/are simultaneously provided to a plurality of parts of the radiotherapy system 100 in order for that to occur. Parts of the radiotherapy system 100 that are so simultaneously triggered may include the magnetron and the electron gun. The simultaneous triggering of various parts of the radiotherapy system 100 will also trigger a number of pulse signals that may be observed in order to assess performance of the radiotherapy system. Examples of such observable pulse signals include: a magnetron current signal, a magnetron voltage signal, an electron gun current signal, an electron gun current signal, an RF forward power signal, an RF reflected power signal, a primary ion chamber current, and a secondary ion chamber current. Such simultaneously triggered signals will have a common nominal pulse width (for example 5 μS) and the nominal pulse width can be used to determine a data capture window for capturing data associated with a given pulse. As one example, the data capture window could be slightly larger than the nominal pulse width so as to ensure that all data associated with a given pulse can be captured.

The radiotherapy system 100 may be operated according to a sequence, or series, of groups of synchronously-triggered pulses, with each different type of pulse signal within the group being triggered synchronously with the same repetition frequency. A data path or route used to obtain and/or transfer a sample of a type of pulse signal data, such as each of the data paths taken by signals obtained from the sources 210a-g through the interfaces 215a-g, may be referred to as a ‘channel’, e.g. ‘the magnetron current channel’, ‘the RF reflected power channel’, and so on.

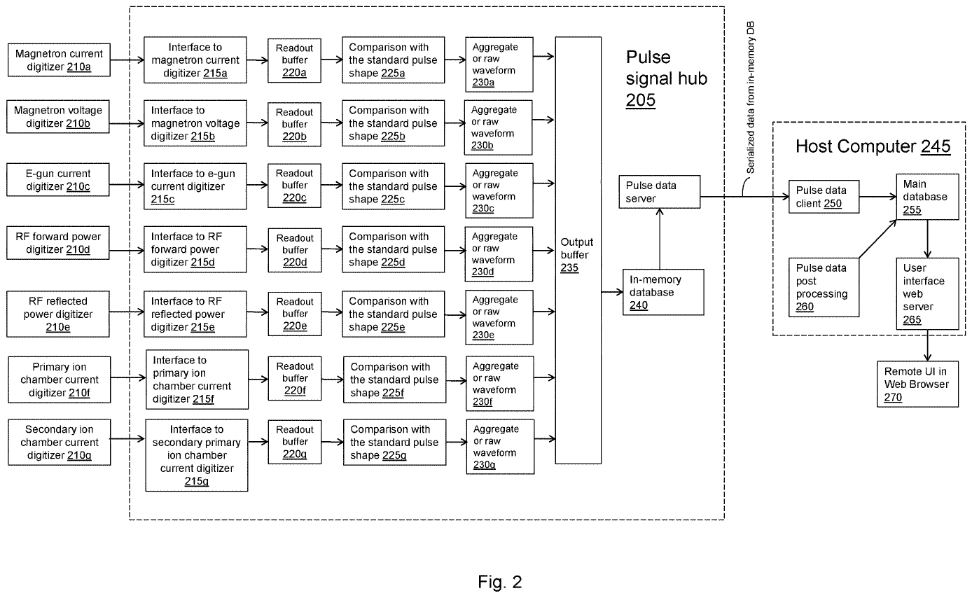

FIG. 2 shows a pulse signal hub 205 that is arranged to record the triggered signals and its configuration and operation will now be explained. Coupled to the magnetron 120 is a magnetron current digitiser 210a which is coupled to a magnetron current digitiser interface 215a. Digitised magnetron current pulse signals that have been recorded by the magnetron current digitiser 210 are received via the magnetron current digitiser interface 215a and conveyed to a corresponding readout buffer 220a as raw signal data. The pulse signal hub 205 then performs at point 225a a comparison of the pulse information from the readout buffer 220a with a standard shape pulse for the magnetron current in order to classify the received magnetron current pulse signal as either being a standard shape or not. The classification may be a similarity comparison, as described further below. When the received magnetron current pulse signal is classified as being a standard shape, there would be little point in storing all of the raw signal data and so at point 230a aggregate pulse information is identified and conveyed to output buffer 235 for subsequent persistent storage. Examples of such aggregate information include: maximum, minimum, average, max, min, time to max, and time to min values; table 1 lists a number of such examples. The aggregate pulse information amounts effectively to a brief description or summary of the raw signal data and so requires less memory than the raw signal data for storage. If however, the received magnetron current pulse signal is classified as not being a standard shape, then there is a deviation from the expected behaviour of the system and so at point 230a all of the raw signal data is conveyed to output buffer 235 along with the additional information for subsequent persistent storage and to enable subsequent analysis of the raw signal data for engineering diagnostics, analysis and/or performance evaluation/prediction. Beneficially, this approach enables detailed analysis in circumstances where an unexpected pulse shape occurs but avoids the need to store the full raw data for all pulses—thereby reducing memory demands.

The additional information that may be saved in addition to the raw/aggregate data may include one or more of: timestamp information; alignment information; information identifying the source of the linac pulse signal; linac configuration information; information identifying the linac; treatment protocol information; index information indexing the linac pulse signal; index information indexing a treatment session during which the linac pulse signal occurred; and a categorisation tag, wherein the categorisation tag comprises an indication as to the normalcy or abnormalcy of the linac pulse signal based on whether or not the shape of the linac pulse signal was classified as being a standard shape or not.

In a manner corresponding to that described above in relation to the magnetron current digitiser 210a and the processing of the raw data therefrom, the pulse signal hub 205 is also arranged to receive signals from: a magnetron voltage digitiser 210b, an e-gun current digitiser 210c, an RF forward power digitiser 210d (which is arranged to digitise the signal produced by an RF forward power measurer—not shown), an RF reflected power digitiser 210e (which is arranged to digitise the signal produced by an RF reflected power measurer—not shown), a primary ion chamber current digitiser 210f, and a secondary ion chamber current digitiser 210g. The RF forward power measurer may be a sensor/detector that is capable of producing a voltage signal proportional to a sensed/detected RF power level. The voltage signal produced may then be digitised using voltage capture/digitisation techniques that will be known to a person skilled in the art. A sensor/detector with appropriate bandwidth, dynamic range, and transient response should preferably be used. In some examples, an additional RF attenuator component may be attached in front of the sensor/detector to protect the sensor/detector from excessive power peaks.

Signals received from the magnetron voltage digitiser 210b, e-gun current digitiser 210c, RF forward power digitiser 210d, RF reflected power digitiser 210e, primary ion chamber current digitiser 210f, and secondary ion chamber current digitiser 210g are received at respective interfaces 215b-g, conveyed to respective readout buffers 220b-g and at respective points 225b-g respective comparisons between the received signals and standard shape pulses for the respective measured quantity are performed in order to classify each received signal as either being a standard shape or not. When a received signal is classified as being a standard shape at respective point 230b-g, aggregate pulse information is identified and conveyed to output buffer 235. If however, the received signal is classified as not being a standard shape, then at respective point 230b-g all of the raw signal data of that received signal is conveyed to output buffer 235. To facilitate parallel data processing features, some or all of features 215a-g to 235 may be implemented by way of Field Programmable Gate Arrays (FPGAs) arranged for parallel processing.

Data from output buffer 235 is conveyed to in-memory database 240 for marshalling in local storage before being provided to pulse data server 242. Pulse data server 242 is arranged to communicate with host computer 245 to provision pulse information from in-memory database 240 to the host computer 245 in a serialised manner. That communication may be performed on a periodic, instantaneous, or other push basis whereby the pulse signal hub 210 pushes its data to the host computer 245 or it may be performed on a pull basis at the instigation of the host computer 245—for example by way of a request received at the pulse data server 242 from a pulse data client 250 of the host computer 245.

Pulse information provided from the pulse signal hub 210 to the host computer 245 is received by a pulse data client 250 which is arranged to convey that information to a main database 255 for persistent storage of data so that, even if the power to the main database 255 is removed, the data will continue to be stored in the main database 255. Main database 255 may be arranged to store not only pulse information received from pulse signal hub 210, but also pulse information either received, for example by way of an ethernet switch, from other pulse signal hubs (not shown) and which may be associated with the same or different linacs or that has been recorded by other means. Accordingly, the main database 255 can store pulse information from different linacs in order to perform large-scale performance comparison and analysis.

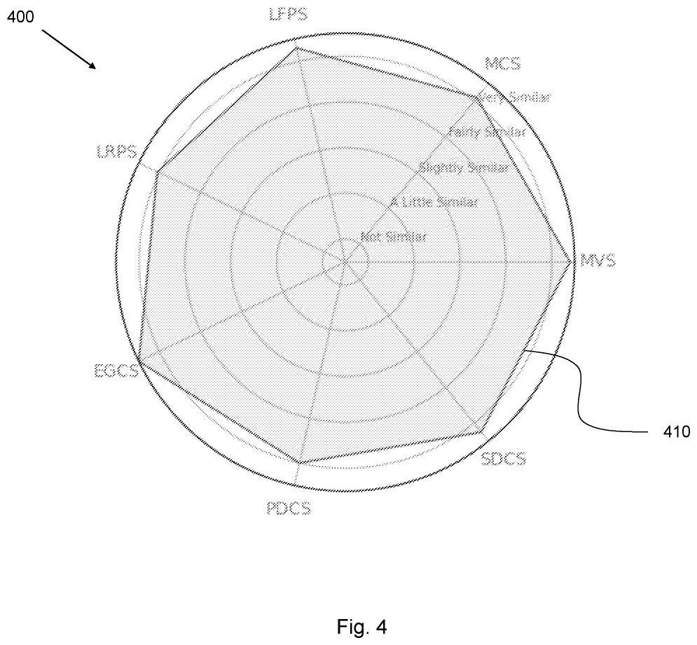

A pulse data post processing unit 260 is communicatively coupled to the main database 255 and may be used to perform analysis of pulse information stored in the main database. Pulse data post processing may occur automatically when pulse data is received, periodically and/or upon user instruction. Examples of activities that may be performed by the pulse data post processing unit 260 include: the identification or prediction of underperformance/failure of a linac or part of it: correlation of linac performance with features of the linac pulse signal samples and/or characterising features; and/or the determination of a health score for a linac based on the database entries associated therewith and the pulse data post processing unit 260 may perform those activities using artificial intelligence and/or a machine learning algorithm. Further examples of pulse data post processing are disclosed below in relation to FIGS. 4 to 6.

The host computer 245 comprises a user interface web server 265 that is communicatively coupled both to the main database 255 and to a remote user interface 270 in a web browser to enable remote interrogation of and interaction with the main database 255.

Alternative arrangements to that shown in FIG. 2 are also considered. For example, not all of the pulse data metrics that are digitised in FIG. 2 may be used and/or additional pulse data metrics may be digitised and processed—for example e-gun voltage. Furthermore the interfaces 215a-g may be distributed across different pulse signal hubs. As one possibility, each hub may interface with up to two different type of digitised pulse data signals and four hubs are used and synchronised. Furthermore, the pulse signal hub 205/host computer 245 split may not be present and instead pulse processing activities such as those carried out on by the pulse data post processing unit 260 may instead be carried out at the pulse signal hub—for example by accessing date stored in the in-memory database 240.

Examples of the data that the database 255 may be arranged to store are set out in table 1. The stored data may be arranged in a single table for each pulse, or may be stored across multiple tables and cross-referenced or joined if needed by reference to an index number of the pulse. The database is also arranged to store a tag (XXX_CAT in table 1 where XXX is a wildcard character or characters) for each pulse data entry to categorise that data following post processing. Examples of such tags include: NORMAL, ABNORMAL TYPE1, ABNORMAL TYPE 2. When a user accesses pulse data in the main database that has been categorised with a tag other than NORMAL, the user may be directed to directly view the pulse waveform data stored at XXX_RAW.

TABLE 1

| Column Name | Data Type | Comments |
|---|---|---|
| TIME | timestamp | absolute time of data capture |
| PULSE_INDEX | integer | The index of the first pulse is 1. |
| SESSION_INDEX | integer | treatment session index |
| MAG_I_MIN | float | magnetron current minimum value |
| MAG_I_MAX | float | magnetron current maximum value |
| MAG_I_AVG | float | magnetron current average value |
| MAG_I_DEV | float | magnetron current deviation value |
| MAG_I_CAT | category | magnetron current category |
| MAG_I_RAW | csv or bin | magnetron current waveform raw data |
| MAG_V_MIN | float | magnetron voltage minimum value |
| MAG_V_MAX | float | magnetron voltage maximum value |
| MAG_V_AVG | float | magnetron voltage average value |
| MAG_V_DEV | float | magnetron voltage deviation value |
| MAG_V_CAT | category | magnetron voltage category |
| MAG_V_RAW | csv or bin | magnetron voltage waveform raw data |
| EGUN_I_MIN | float | e-gun current minimum value |
| EGUN_I_MIN | float | e-gun current maximum value |
| EGUN_I_AVG | float | e-gun current average value |
| EGUN_I_DEV | float | e-gun current deviation value |
| EGUN_I_CAT | category | e-gun current category |
| EGUN_I_RAW | csv or bin | e-gun current waveform raw data |
| FWD_P_MIN | float | RF signal forward power minimum value |
| FWD_P_MAX | float | RF signal forward power maximum value |
| FWD_P_AVG | float | RF signal forward power average value |
| FWD_P_DEV | float | RF signal forward power deviation value |
| FWD_P_CAT | category | RF signal forward power category |
| FWD_P_RAW | csv or bin | RF signal forward power waveform raw data |
| RFL_P_MIN | float | RF signal reflected power minimum value |
| RFL_P_MAX | float | RF signal reflected power maximum value |
| RFL_P_AVG | float | RF signal reflected power average value |
| RFL_P_DEV | float | RF signal reflected power deviation value |
| RFL_P_CAT | category | RF signal reflected power category |
| RFL_P_RAW | csv or bin | RF signal reflected power waveform raw data |
| PRI_ION_I_MIN | float | primary ion chamber current minimum value |
| PRI_ION_I_MAX | float | primary ion chamber current maximum value |
| PRI_ION_I_ACC | float | primary ion chamber current accumulated value |
| PRI_ION_I_DEV | float | primary ion chamber current deviation value |
| PRI_ION_I_CAT | category | primary ion chamber current category |
| PRI_ION_I_RAW | csv or bin | primary ion chamber current waveform raw data |
| SEC_ION_I_MIN | float | secondary ion chamber current minimum value |
| SEC_ION_I_MAX | float | secondary ion chamber current maximum value |
| SEC_ION_I ACC | float | secondary ion chamber current accumulated value |
| SEC_ION_I DEV | float | secondary ion chamber current deviation value |
| SEC_ION_I_CAT | category | secondary ion chamber current category |
| SEC_ION_I_RAW | csv or bin | secondary ion chamber current waveform raw data |

Additionally or alternatively, in situations where very large amounts of data storage are available, the pulse signal hub may be arranged to pipe data from the readout buffers 220a-g directly to output buffer 235 in order to enable persistent storage of all pulse signal data produced by a linac. As an example in such a case with a linac life cycle of 5000 hours, a pulse repetition frequency of 275, a pulse signal sampling rate of 100 M samples/second, with 2 bytes per sample 8 bytes per timestamp and 4 bytes per index and with a 5 μS sampling window and the seven types of pulse signal shown in FIG. 2 being recorded, 5000*3600*275*(5*10−6*100*106*(2+8+4))*7=~35 T bytes would be needed—more than the largest hard disk drive that is currently commercially available.

As further examples, the database 255 may be arranged to additionally store data relevant to the particular treatment delivery that the pulse occurred within—such as the angle of irradiation in order that such information be available for subsequent analysis of that pulse data.

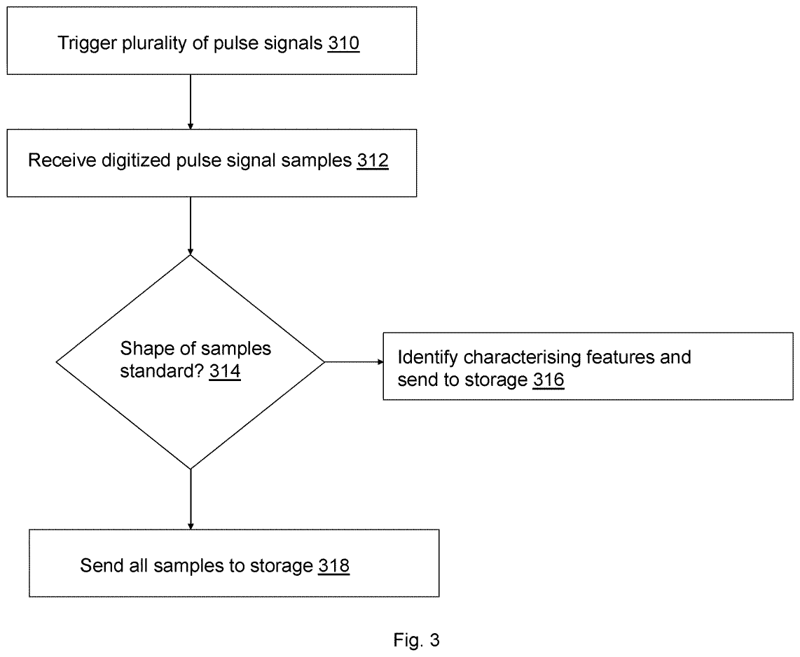

FIG. 3 shows a flowchart of a method of processing pulse signal information of a radiotherapy linear accelerator. At point 310, the method starts when a plurality of pulse signals are simultaneously triggered. Steps 312 to 318 are then performed in parallel for each of the pulse signals. At point 312 the pulse signal hub 205 receives digitized pulse signal samples before the pulse signal samples are analysed at step 314 to classify the shape of the pulse signal samples as having either a standard shape or not. In the event that the pulse signal samples are classified as having a standard shape then, at step 316, one or more characterising features are determined from the linac pulse signal samples as aggregate pulse information characterising the linac pulse signal and sent for persistent storage along with additional information. If however, the pulse signal samples are classified as not having a standard shape then, at step 318, all of the pulse signal samples for that pulse signal are sent to be persistently stored in memory as the raw data for that pulse signal and are sent along with additional. Data that is sent for persistent storage will be stored first in the in-memory database 240 and preferably the steps of FIG. 3 up to and including at least storage in the in-memory database 240 are performed in real time such that the time between step 312 and storage in the in-memory database 240 is less than one over the Pulse Repetitive Frequency (PRF)—the time between temporally adjacent pulses or pulse frame.

As used herein, the ‘shape’ of a pulse signal or a pulse signal sample may refer to the curve of the pulse signal or pulse signal sample when plotted as pulse signal amplitude versus time. Alternatively, or additionally, the ‘shape’ may refer to a frequency domain representation of a pulse, such as a Fourier transform, or fast Fourier transform, of a pulse signal sample. Alternatively or additionally, the ‘shape’ of the pulse signal may be a parameterisation of pulse signal sample data, and may refer to the waveform of the pulse in part or in its entirety (such as a square or sawtooth pulse having an amplitude slope and/or duration). A parameterisation of the pulse signal sample data may alternatively or additionally include an average and/or mean value, and/or a standard deviation value, of the pulse signal sample. It will be appreciated that a combination of the above examples may be used in order to determine the ‘shape’ of a pulse signal or pulse signal sample. The terms ‘sampled’ or ‘captured’ may be used interchangeably to describe the process of obtaining the pulse signal shape.

The analysis performed at step 314 to classify the shape of the pulse signal samples as having either a standard shape or not may be performed in a number of different manners. For example, the similarity to a measured or ideal standard pulse may be measured using a similarity measure such as the sum of squared differences, cosine similarity, partial curve mapping, an area-based similarity measure, or a measure of mutual information. The measured or ideal standard pulses may be different for pulses that have different origins, so the measured or ideal standard pulses of a first pulse originating from a first source (such as the magnetron voltage digitiser) may differ from of a second pulse originating from a different source (such as a magnetron current digitiser, or a primary ion chamber current digitiser). Alternatively or additionally, the pulse may be classified using a clustering algorithm—for example a C or K-means clustering algorithm. As a further option, artificial intelligence/a machine learning algorithm, such as a neural network, may be trained on existing pulse information for the same or other linacs—possibly along with information about failures or required maintenance for that linac—in order to enable the machine learning algorithm to classify each pulse.

As outlined above, multiple pulse signals are synchronously triggered during linac operation, such as the magnetron current signal, and the e-gun current signal. As one possibility, seven different types of pulse signal are synchronously triggered for a linac, wherein one type of pulse signal is the magnetron current signal, another type of pulse signal is the e-gun current signal, and so on as in FIG. 2. However, it will be appreciated that not all of the seven types of pulse signal need be triggered/analysed in order to provide useful information about the linac. In some examples, only one type of pulse signal may be used by the linac or sampled for analysis. In the examples herein, seven types of synchronously-triggered pulse signal are considered merely as an illustrative example, and the methods and systems disclosed herein may be used with or applied to any number or combination of pulse signal types, include a single type of pulse signal.

FIG. 4 shows an example output of a method of processing pulse signal information of a radiotherapy linear accelerator, such as the methods disclosed herein. The output is a radar chart 400.

The radar chart 400 of FIG. 4 has seven axes, with each axis corresponding to a different type of pulse signal involved in linac operation. In other examples, a different number of types of pulse signal may be used as variables or parameters, and a corresponding number of axes may be used for the radar chart 400. The similarity of each of a given plurality of synchronously-triggered pulse signals is plotted as a plot 410 on the radar chart 400. Such a radar chart 400 can be used to determine and/or represent a health score for a linac. For example, for some linacs, the main beam generation module deliverable consists of the following seven types of pulse signals, each of which is represented on a respective axis of radar chart 400:
1. Magnetron Voltage, with a similarity-to-standard-shape metric abbreviated as MVS;
2. Magnetron Current, with a similarity-to-standard-shape metric abbreviated as MCS;
3. E-Gun Current, with a similarity-to-standard-shape metric abbreviated as EGCS;
4. Linac Forward Power to RF cavity, with a similarity-to-standard-shape metric abbreviated as LFPS;
5. Linac Reflected Power from RF cavity, with a similarity-to-standard-shape metric abbreviated as LRPS;
6. Primary Dosimetry Current, with a similarity-to-standard-shape metric abbreviated as PDCS;
7. Secondary Dosimetry Current, with a similarity-to-standard-shape metric abbreviated as SDCS.

Each The standard shapes of each of these pulse signals can be manually crafted or directly taken from “golden” samples of a well-performing system. Properly working linac beam generation modules all produce an alike radar chart, as long as following 7 conditions are met:
1. high similarity of magnetron voltage pulse signal to standard shape;
2. high similarity of magnetron current pulse signal to standard shape;
3. high similarity of e-gun current pulse signal to standard shape;
4. high similarity of forward power pulse signal to standard shape;
5. high similarity of reflected power pulse signal to standard shape;
6. high similarity of primary dosimetry current pulse signal to standard shape;
7. high similarity of secondary dosimetry current pulse signal to standard shape.

It will be appreciated that in other examples, a different set of parameters and signals may be considered and there may be more or less than seven criteria. Whichever criteria and number of signals are used, if one of the criteria is not met, e.g. one of the types of pulse signal is non-standard, or abnormal, the radar chart will provide an effective indication to the user. The user can thus be alerted to a possible error with the radiotherapy system.

The radar chart 400 may be produced based on analysis of data from either, or both of, the in-memory database 240 or the main database 255. Likewise, a particular radar chart may be stored in the in-memory database 240 and/or the main database 255. For example, if an abnormal waveform is detected and stored, the corresponding radar chart may be stored with it. The radar chart may be displayed on a screen to the user during linac operation and updated in real-time. Alternatively, the radar chart may be displayed and/or updated only in the event of an error.

The approaches disclosed herein may also be used alongside, or as part of, approaches for centrally-logging pulse signal abnormal waveforms. In some examples, pulse signal abnormalities from multiple linacs in different locations can be centrally logged and analysed at a central system.

When a beam generation module is deemed to be working abnormally according to the approaches disclosed above, an operator or on-site user may not know what the underlying issue is without further analysis of the abnormality. In order to provide a further determination and classification of errors, abnormal pulse signals may be further analysed and machine learning approaches such as unsupervised clustering may be used. However, localised, individual linac operation sites do not typically generate enough data for an unsupervised clustering analysis, nor for labelling based on best R&D engineering judgment and/or tagging based on service engineering experience. Therefore, it is beneficial to use a centralised approach in which a central system receives data from multiple linacs, which can be used to train a model to analyse abnormal waveforms and determine and/or indicate what type of error and/or failure has occurred.

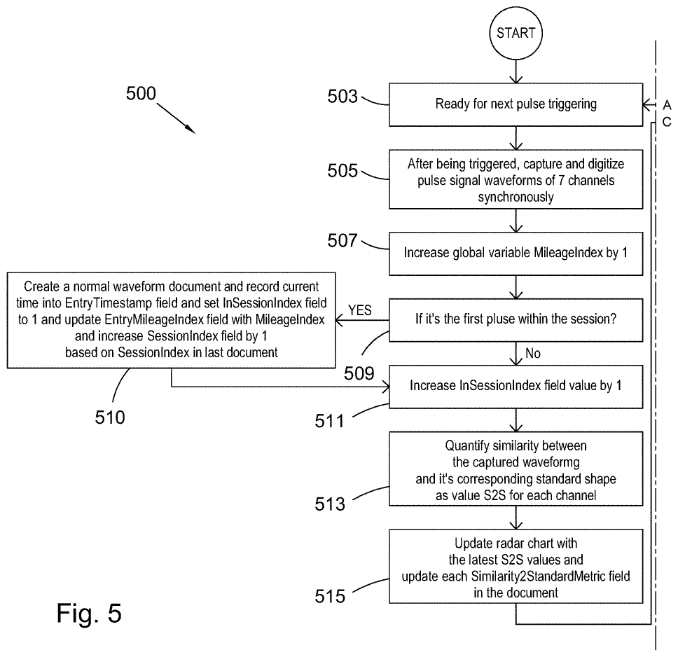

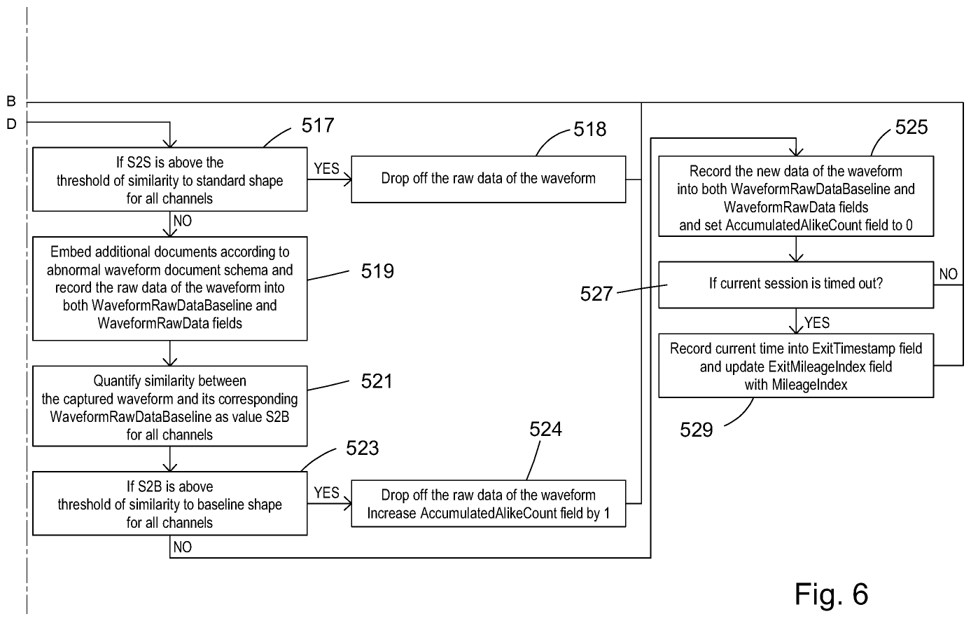

FIGS. 5 and 6 show a flowchart of a method 500 of logging pulse signal abnormal waveforms that may be used with such a centralised learning approach. FIG. 6 is a continuation of FIG. 5, with the point A of FIG. 5 corresponding to the point B of FIG. 6, and the point C of FIG. 5 corresponding to the point D of FIG. 6, such that there is a continuous connection from box 515 to box 517, and likewise a continuous connection between boxes 518, 524, 527, 529 and box 503. The method 500 records errors from individual local linacs in a manner that is highly suitable for sending to a remote data centre, or server.

Raw data of abnormal waveforms from a particular linac can be deemed as a special form of error log. Such an error log is firstly stored at the local site (using the approaches described herein, such as by storing it at the main database 255) and then sent to the remote site for diagnosis and central model training.

Preferably, storing the error log in a database, as compared with a file system, provides easier organization and greater query power. The data model of the error log is document-oriented. In the context of databases, a document is essentially a set of property names and their values. The values can be simple data types, such as strings, numbers, dates, arrays, or even other documents (preferably JSON documents). The database stores data in collections of documents. Each document in a collection can have a completely different structure.

At a first step 503 of the method 500, the system is readied for a pulse triggering event. The first step 503 may be equivalent to the first step 310 of the method of FIG. 3. A document is initially created using a normal waveform schema such as that shown in Table 2. In Table 2, only the MagnetronVoltageChannel and MagnetronCurrentChannel sub-properties are depicted, but it will be appreciated that corresponding sub-properties may be recorded for each Channel.

TABLE 2

```
EntryTimestamp: 1646209052733
EntryMileageIndex: 1000
SessionIndex: 10
InSessionIndex: 1001
ExitMileageIndex: 2000
ExitTimestamp: 1646209435702
MagnetronVoltageChannel
  StandardShapeName: magnetron_voltage_standard_shape
  SimilarityMeasureType: partial_curve_mapping
  Similarity2StandardThreshold: 8.0
  Similarity2StandardMetric: 9.2
MagnetronCurrentChannel
  StandardShapeName: magnetron_current_standard_shape
  SimilarityMeasureType: area_method
  Similarity2StandardThreshold: 9.0
  Similarity2StandardMetric: 9.5
EGunCurrentChannel
ForwardPowerChannel
ReflectedPowerChannel
PrimaryDosimetryCurrentChannel
SecondaryDosimetryCurrentChannel
```

After being triggered, the pulse signal waveforms of the multiple (in this example, seven) channels are captured and digitized as a next step 505, which may be in accordance with approaches disclosed herein, such as the approach of the second step 312 of the method of FIG. 3.

At a next step 507, a global variable MileageIndex is increased by 1 at the local site in order to keep a record of the overall number of pulse events that have been triggered by the system over its lifetime.

At a next step 509, the method determines whether the set of synchronous pulses are the first pulse set within a session. Herein, a session is a key concept that means a set of consecutive pulses within a limited time interval. If the time interval between two consecutive pulses exceeds the largest permitted interval, e.g. SESSION_TIMEOUT=1 second, then the previous pulse is considered as the last pulse of the previous session, while the latter pulse is considered as the first pulse of the latter session. In some examples, the largest permitted time interval accounts for the possibility of a missing pulse. In an example, only a document that has its both WaveformRawData and ExitTimestamp fields recorded, which indicates that the data captured is complete and ready for failure diagnosis, is to be processed by the central model. Each document will only contain data that has been captured within a specific session.

If the pulse set is determined to be the first pulse set within the session, a normal waveform document is created 510 for the session, the current time is recorded into the EntryTimestamp field, and the InSessionIndex field is set to 1. The EntryMileageIndex is recorded according to the current MileageIndex at the start of the session, and the SessionIndex field, which counts global sessions, is updated.

At a next step 511, the InSessionIndex field value is then set to or increased by 1 in order to record the position of the pulse in the sequence of pulses forming the session.

At a next step 513, the similarity between the sampled pulse signal waveform and its corresponding standard shape is determined, and a resultant value labelled ‘S2S’ is determined for each channel. In some examples, this step 513 comprises the classification step 314 of the method of FIG. 3.

At a next step 515, a radar chart such as is shown in FIG. 4 is created, or is updated with the S2S values and a Similarity2StandardMetric field is recorded in the document with the same. The method 500 then determines 517 for each channel whether the S2S value for a pulse is above a threshold of similarity to the standard shape. If the S2S value indicates that the pulse signal is sufficiently similar, the raw data of the pulse waveform is dropped off/discarded 518. ‘Dropping off’ the raw data of the pulse waveform may be considered as, and may comprise, a form of data compression. With the systems and approaches disclosed herein, it is not necessary to record data for each similar pulse shape because the pulse signals are alike in shape under most normal conditions. Having recorded the raw data of a first pulse of a particular pulse type, ‘dropping’ or ‘dropping off’ similar data of subsequent pulses by compressing (or discarding entirely) the data means that the storage space required for the error log is reduced. Furthermore, the complexity of subsequent data processing is reduced and the recorded data may be queried more quickly. ‘Dropping’ or discarding data in this way beneficially enables a form of data compression of the error log that does not notably reduce the accuracy of the error log in reflecting the true working state of the system. In other examples, the full data of further pulse signals may indeed be recorded and stored.

Due to the error log being modelled as a document, new properties can be added dynamically. An example abnormal waveform schema, which is a modification of the schema of Table 2, is set out in Table 3. If the abnormal waveform similarity metric is below the predefined Similarity2StandardThreshold when classified, i.e. the pulse signal is abnormal, the following additional fields and/or nodes (i.e. sub-documents) are added to the document to aid recording the raw data of the abnormal waveform and to assist further analysis:
AccumulatedAlikeCount field;
DiagnosticResult node;
WaveformRawDataBaseline node for each channel;
WaveformRawData node for each channel;
Similarity2BaselineThreshold field for each channel;
Similarity2BaselineMetric field for each channel.

For simplicity, only 4 elements are listed under WaveformRawData and WaveformRawDataBaseline nodes in the example of Table 3. In other examples, the elements can be adjusted according to the actual sample point quantity, e.g. 1024.

TABLE 3

```
EntryTimestamp: 1646209052733
EntryMileageIndex: 1000
SessionIndex: 10
InSessionIndex: 1001
AccumulatedAlikeCount: 80
ExitMileageIndex: 2000
ExitTimestamp: 1646209435702
DiagnosticResult
  ErrorType: Magnetron load mismatch
  SeverityLevel: High
  ServiceAdvice: Please contact Elekta service
MagnetronVoltageChannel
  StandardShapeName: magnetron_voltage_standard_shape
  SimilarityMeasureType: partial_curve_mapping
  Similarity2StandardThreshold: 8.0
  Similarity2StandardMetric: 7.2
  Similarity2BaselineThreshold: 9.0:
  Similarity2BaselineMetric: 9.9
  WaveformRawDataBaseline
    [0]: 123.4
    [1]: 101.0
    [2]: 120.1
    [3]: 108.9
  WaveformRawData
    [0]: 123.3
    [1]: 101.1
    [2]: 120.0
    [3]: 108.9
MagnetronCurrentChannel
  StandardShapeName: magnetron_current_standard_shape
  SimilarityMeasureType: area method
  Similarity2StandardThreshold: 9.0
  Similarity2StandardMetric: 9.2
  Similarity2BaselineThreshold: 9.0
  Similarity2BaselineMetric: 9.8
  WaveformRawDataBaseline
    [0]: 213.0
    [1]: 213.1
    [2]: 212.4
    [3]: 212.2
  WaveformRawData
    [0]: 213.1
    [1]: 213.2
    [2]: 211.5
    [3]: 211.9
EGunCurrentChannel
```

If the S2S value indicates 517 that the pulse signal is not sufficiently similar to the standard, the method proceeds to a next step 519, at which the document is embedded with additional properties according to an abnormal waveform schema such as that of table 3, and the raw data of the waveform is recorded into WaveformRawDataBaseline and WaveformRawData fields. The pulse signal is hence associated with the session.

From the preceding steps 517, 519, only an abnormal waveform, i.e. a waveform deviating too much from the standard shape, has its raw data recorded in a document.

The method proceeds to another comparison/classification step 521. In this step 521, the similarity of the sampled/captured waveform is compared to its corresponding WaveformRawDataBaseline as an ‘S2B’ value. This step 521 is performed for at least one channel.

The next step 523 comprises determining whether S2B is above the threshold of similarity to a baseline shape for the channels of interest in order to classify the pulse. If the S2B value indicates that the current pulse is similar to the WaveformRawDataBaseline, i.e. that the current pulse is similar to a previously-recorded abnormal pulse from within the session, then the raw data of the waveform is not associated with the session, or recorded in the session document.

Instead, an AccumulatedAlikeCount field is increased/incremented by 1, which provides a count and/or of the number of similar abnormal pulses within a session. The count of the number of similarly abnormal pulses within a session is hence associated with the session and the raw data of an abnormal pulse.

The method thus provides a highly effective approach to data storage, in which a very high number of pulses can be analysed and an error log is produced that stores highly informative records of the error with a relatively very small amount of memory.

If it is determined that a pulse is abnormal but is not similar to a previous abnormal pulse in the same session, the method proceeds to a step 525 at which the raw data of the most recent pulse is recorded into the WaveformRawDataBaseline and WaveformRawData fields. In some examples, the raw data of the most recent abnormal pulse may replace the raw data of the previous abnormal pulse within the WaveformRawDataBaseline and WaveformRawData fields (in other words, the previous data may be overwritten with the raw data of the most recent abnormal pulse) and the AccumulatedAlikeCount is set to zero. Hence, in those examples, the last-occurring type of abnormal pulse within a session is considered to represent the most significant error-type or failure-type of the session. In other examples, the raw data of each type of abnormal pulse signal within a session is recorded, and respective AccumulatedAlikeCount counters may be maintained for each type of abnormal pulse signal.

Next, the method 500 involves checking 527 whether the session has timed out by using a predetermined value of an expected time interval between pulses. The predetermined value thus represents a predetermined threshold. If the session has not timed out, the method repeats by arranging the system to be ready 503 for the next pulse triggering. If the session has timed out, the method 500 proceeds to record 529 the current time into the ExitTimestamp field and update the ExitMileageIndex field with the MileageIndex value. Hence, an error log for the session is developed.

By using the expected time interval in this way, a session can be identified from the record of the pulse data itself. The session comprises a sequence of pulse events, wherein each pulse event comprises a plurality of synchronously triggered linac pulse signals. Identifying the session comprises estimating a time interval between successive pluralities of synchronously triggered linac pulse signals, wherein a start and/or end of the session is established when the time interval between two successive pulse events exceeds a predetermined threshold.

Each session corresponds to a separate document, and vice versa. It will be appreciated that other file formats and database types may be used in place of a document.

The error log may be sent to a central system or database, along with error logs from other linacs, for further analysis with a central model. At the central model side, the received document may go through the following two-stage classification process to get a diagnostic result:
1. The first stage classifies the waveform error type based on the seven Similarity2StandardMetric (S2S) values and, optionally, other scalar properties, such as SessionIndex, AccumulatedAlikeCount, etc.
2. The second stage classifies the severity level of waveform abnormality based on the raw data of the individual waveforms.

The first stage classifier model can be trained by knowledgeable R&D engineers by correlating failure modes with their corresponding waveforms properties. The second stage classifier deals with the time series data, and may utilise automatic feature extraction.

For both stages, after an unsupervised clustering step, data labelling can be done by a collaboration of knowledgeable R&D engineers and experienced service engineers. Thereafter, the central model can be trained using supervised machine learning algorithms to enable it to make predictions, i.e., diagnostic results. Additionally, based on the diagnostic results, service advice could be derived and attached to the document, which may be returned to the linac operator.

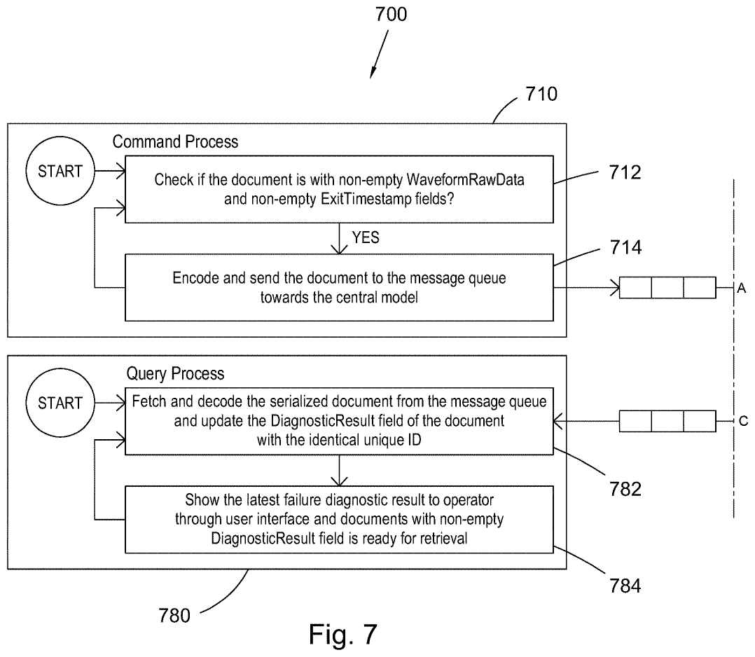

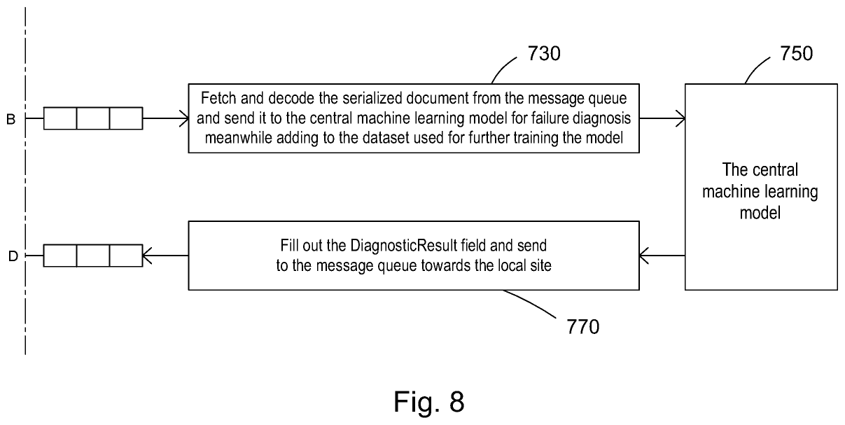

FIGS. 7 and 8 show a flowchart of a method 700 of interacting with such a central model for error logging. FIG. 8 is a continuation of FIG. 7, with the point A of FIG. 7 corresponding to the point B of FIG. 8, and the point C of FIG. 7 corresponding to the point D of FIG. 8, such that there is a continuous connection from box 714 to box 730, and likewise a continuous connection from box 770 to box 782. The sequences of three rectangular boxes between box 714 and box 730, and between box 770 and box 782, represent the transmission of data. The central model may be implemented on a remote data centre, server, or system. In some examples, the central model and/or the remote data centre may correspond to, or host, the main database 255.

The local system at the site of the linac, such as host computer 245, runs a command process 710. The command process 710 comprises checking if an error log such as that produced by the method 500 of FIGS. 5 and 6 has non-empty WaveformRawData and ExitTimestamp fields. If so, the error log, which in this example is a document, is considered as a complete error log, and is encoded 714 and sent as part of a message queue towards the central model (a step which may optionally also include serializing the document). The remote data centre then fetches 730 and decodes the serialized document from the message queue and sends it to the central machine learning model 750 for failure diagnoses, as well as adding the document to the dataset used to further train the central machine learning model 750.

The central machine learning model 750 provides a diagnosis of the error, which is added 770 to a DiagnosticResult field of the document and sent in a message queue towards the local site.

The local site then runs a query process 780, in which it fetches 782 and decodes the document from the message queue and updates its local copy of the document with the entry of the DiagnosticResult field. The local operator can then be shown 784 the diagnosed cause of failure, or error, through a user interface, and take appropriate action. Hence, a greatly improved troubleshooting service can be provided to local operators by pooling and analysing error reports at the central machine learning model 750. Such an approach is enabled by an efficient approach to handling the vast amounts of data required in order to accurately monitor for errors in linac operation.

The approaches described herein provide a number of benefits. In particular:
The described approaches give the user a continuous historical context for the linac in question which can help them to understand the extent of the impact of an issue with that linac that is indicated by an abnormal pulse shape and how it evolved over time—which may be used to inform future proactive maintenance aimed at avoiding future repetition.
The recording of pulse signal information, either in aggregate or raw form, for all pulses throughout the product lifecycle of a linac helps to track the specific usage of that machine in a more informed manner than a single rough HT (High Tension) hour indicator.
The large amounts of historical data gathered can help machine learning algorithms automatically recognize potential patterns including but not limited to:
1. issues in a given treatment session—for example an issue occurring or becoming apparent during the current treatment session requiring issuance of an alarm and/or an immediate shutdown,
2. anomaly trends, e.g. magnetron RF pulse missing, waveguide arcing, unexpected pulse histograms, etc,
3. correlations between pulse waveform features (X) and linac performance, e.g. dose rate settling time and stability,
4. indications that preventative maintenance should be carried out,
5. power degradation issues (and consequent power adjustments to compensate therefor).
Users can easily and quickly search for waveforms of interest from the main database 255 based on waveform features and/or categories. Typical pulse symptoms for problematic linacs may be accumulated to facilitate rapid future diagnosis of issues with a given linac.
Based on the main database 255 content, a linac health scoring system may be employed and a given linac scored to provide a quick indication as to whether or not that linac is operating in a healthy state.
A centralised approach, in which errors from multiple differently-situated local linac devices are recorded and analysed at a remote system, allows for many error types to be analysed, a central model to be trained based on that, and for improved feedback to be provided to operators.

Recording information according to an abnormal waveform schema like that of Table 3 provides the following benefits:
1. EntryTimestamp and/or EntryMilageIndex (the absolute pulse index since linac machine installation) helps to compose a unique ID to search out the right document.
2. SessionIndex (the absolute session index since linac machine installation) helps to identify high tension cycles experienced by linac since installation. This may be a relevant feature to the machine learning model.
3. InSessionIndex should always equal ExitMilageIndex −EntryMilageIndex. This may be used to check document data integrity.
4. (ExitTimestamp−EntryTimestamp −SESSION_TIMEOUT)/(ExitMilageIndex−EntryMilageIndex)
5. should equal the PRF (pulse repetitive frequency) setting. If not, a missing pulse must have occurred. This is a common failure mode that can be identified by the document itself.
6. AccumulatedAlikeCount shows how many similar abnormal waveforms were consistently detected within a session. This could be a relevant feature to the machine learning model.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer-readable media or, more generally, a computer program product. The computer-readable media may be transitory or non-transitory. The one or more computer-readable media could be, for example, an electronic, magnetic, optical, electromagnetic, infrared, or semiconductor system, or a propagation medium for data transmission, for example for downloading the code over the Internet. Alternatively, the one or more computer-readable media could take the form of one or more physical computer-readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

Methods, apparatus and systems are disclosed for analysing linac pulse signal samples to determine whether their shape is a standard one or not and, when the shape is not saving the raw data of the linac pulse signal samples and, when the shape is a standard one, saving aggregated data that describes the linac pulse signal using less memory than the raw data.

Those skilled in the art will recognise that a wide variety of modifications, alterations, and combinations can be made with respect to the above described examples without departing from the scope of the disclosed concepts, and that such modifications, alterations, and combinations are to be viewed as being within the ambit of the disclosed concepts.

Those skilled in the art will also recognise that the scope of the invention is not limited by the examples described herein, but is instead defined by the appended claims.

1. A method of processing pulse signal information of a radiotherapy linear accelerator, linac, the method comprising, for a plurality of synchronously triggered linac pulse signals:
receiving linac pulse signal samples produced by sampling a linac pulse signal;
analysing the linac pulse signal samples to classify a shape of the linac pulse signal as either being a standard shape or not being a standard shape;
when the shape of the linac pulse signal is classified as not being a standard shape, sending the linac pulse signal samples to be persistently stored in memory;
when the shape of the linac pulse signal is classified as being a standard shape, sending to be persistently stored in memory one or more characterising features determined from the linac pulse signal samples as characterising the linac pulse signal, wherein an amount of memory required to store the one or more characterising features is less than an amount of memory required to store the linac pulse signal samples.

2. The method of claim 1, wherein a first one or more standard shapes for a first pulse signal originating from a first source within the linac differs from a second one or more standard shapes for a second pulse signal originating from a second source within the linac.

3. The method of claim 1, wherein the analysing of the linac pulse signal samples of each of the plurality of synchronously triggered linac pulse signals is performed in parallel.

4. The method of claim 1, wherein the method is performed within a time window corresponding to an expected interval between consecutive linac pulse signals.

5. The method of claim 4, wherein the time window is 5 μS or less.

6. The method of claim 1, further comprising, for a plurality of synchronously triggered linac pulse signals, after analysing the linac pulse signal samples, marshalling the linac pulse signal samples and/or the one or more characterising features in local memory prior to sending them to be persistently stored in memory.

7. The method of claim 1, comprising using an artificial intelligence or machine learning algorithm to classify the shape.

8. The method of claim 1, further comprising, for the plurality of synchronously triggered linac pulse signals, sending to be persistently stored in memory additional information relating to the linac pulse signal.

9. The method of claim 8, wherein the additional information comprises one or more of: timestamp information; information identifying a source of the linac pulse signal; linac configuration information; information identifying the linac; treatment protocol information; index information indexing the linac pulse signal; index information indexing a treatment session during which the linac pulse signal occurred; and a categorisation tag, wherein the categorisation tag comprises an indication as to a normalcy or abnormalcy of the linac pulse signal based on whether or not the shape of the linac pulse signal was classified as being a standard shape or not.

10. The method of claim 1, wherein the plurality of synchronously triggered linac pulse signals are pulse signals of two or more of: magnetron current; magnetron voltage; electron gun current: Radio Frequency, RF, signal forward power; RF signal reflected power; primary ion chamber current; and secondary ion chamber current.

11. The method of claim 1, further comprising, for each of the plurality of synchronously triggered linac pulse signals, determining the one or more characterising features from the linac pulse signal samples.

12. The method of claim 1, wherein the one or more characterising features comprise one or more of: a maximum value, a minimum value, an average value, and a deviation value.

13. A method comprising receiving, and persistently storing as database entries in a database, linac pulse signal samples and/or one or more characterising features sent according to the method of claim 1, as performed for a first linac.

14. The method of claim 13 further comprising, receiving and persistently storing as database entries in the database, linac pulse signal samples and/or one or more characterising features sent according to the method of claim 1, as performed for a second or further linac.

15. The method of claim 13, further comprising analysing the database entries to at least one of:
identify or predict underperformance and or failure of a linac or part thereof;
correlate linac performance with features of the linac pulse signal samples and/or characterising features; or
determine a health score for a linac based on the database entries associated therewith.

16. The method of claim 15, wherein the analysing the database entries is performed at least in part by an artificial intelligence or machine learning algorithm.

17. A method of processing pulse signal information of a radiotherapy linear accelerator, linac, the method comprising:
analysing a sample of a first linac pulse signal to classify a shape of the first linac pulse signal as either being a standard shape or not being a standard shape;
when the shape of the first linac pulse signal is classified as not being a standard shape, recording raw data of the first linac pulse signal and associating the raw data of the first linac pulse signal with a session;
classifying a subsequent linac pulse signal as either being a similar shape to the first linac pulse signal or not;
if the shape of a subsequent linac pulse signal is classified as being similar to the shape of the first linac pulse signal, incrementing a count of a number of linac pulse signals within the session that are not a standard shape.

18. The method of claim 17, wherein the first linac pulse signal is one of a first plurality of synchronously triggered linac pulse signals, and the subsequent linac pulse signal is one of a second plurality of synchronously triggered linac pulse signals.

19. The method of claim 17, the method further comprising:
analysing a sample of the subsequent linac pulse signal to classify the shape of the subsequent linac pulse signal as either being a standard shape or not being a standard shape; and
if the shape of the subsequent linac pulse signal is classified as not being similar to the shape of the first linac pulse signal, and is classified as not being a standard shape,
recording the raw data of the subsequent linac pulse signal and associating the raw data of the subsequent linac pulse signal with the session.

20. The method of claim 19, wherein recording the raw data of the subsequent linac pulse signal comprises replacing the recording of the raw data of the first linac pulse signal associated with the session with a recording of the raw data of the subsequent linac pulse signal.

21. The method of claim 17, further comprising:
identifying the session, the session comprising a sequence of pulse events, wherein each pulse event comprises a plurality of synchronously triggered linac pulse signals;
wherein identifying the session comprises estimating a time interval between successive pluralities of synchronously triggered linac pulse signals, wherein a start and/or end of the session is established when the time interval between two successive pulse events exceeds a predetermined threshold;
and wherein associating data with the session comprises including the data in an error log for the session.

22. The method of claim 18, further comprising at least one of:
(A) wherein the first linac pulse signal is one of a first plurality of synchronously triggered linac pulse signals, and the subsequent linac pulse signal is one of a second plurality of synchronously triggered linac pulse signals;
(B) analysing a sample of the subsequent linac pulse signal to classify the shape of the subsequent linac pulse signal as either being a standard shape or not being a standard shape; and if the shape of the subsequent linac pulse signal is classified as not being similar to the shape of the first linac pulse signal, and is classified as not being a standard shape, recording the raw data of the subsequent linac pulse signal and associating the raw data of the subsequent linac pulse signal with the session;
(C) recording the raw data of the subsequent linac pulse signal comprises replacing the recording of the raw data of the first linac pulse signal associated with the session with a recording of the raw data of the subsequent linac pulse signal; or
(D) identifying the session, the session comprising a sequence of pulse events, wherein each pulse event comprises a plurality of synchronously triggered linac pulse signals: wherein identifying the session comprises estimating a time interval between successive pluralities of synchronously triggered linac pulse signals, wherein a start and/or end of the session is established when the time interval between two successive pulse events exceeds a predetermined threshold; and wherein associating data with the session comprises including the data in an error log for the session.

23. An apparatus or system arranged to perform the method of claim 1.

24. A computer readable medium or signal carrying machine readable instructions that, when executed on one or more processors, cause the method of claim 1 to be carried out.
